(19) United States
(12) Patent Application Publication
(10) Pub. No.: US 2025/0090845 A1
(43) Pub. Date: Mar. 20, 2025

(54) Gastric Electrical Stimulation System

(71) Applicant: National Chin-Yi University of Technology, Taichung City (TW)

(72) Inventor: Jian-Xing WU, Taichung City (TW)

(21) Appl. No.: 18/514,013

(22) Filed: Nov. 20, 2023

(30) Foreign Application Priority Data
Sep. 14, 2023 (TW) 112135062

(51) Int. Cl. A61N 1/36; A61N 1/02; A61N 1/05

(52) U.S. Cl. CPC A61N 1/36007; A61N 1/025; A61N 1/0507

(57) ABSTRACT

The present invention discloses a gastric electrical stimulation system that integrates gastric electrical stimulation capsule technology into ultrasonic capsules. By using ultrasonic stimulation, piezoelectric effects are induced in materials. Through electrodes and pulse generators, the gastric tissue is electrically stimulated, effectively alleviating the symptoms of gastroparesis.

### BACKGROUND OF THE INVENTION

### 1. Field of the Invention

The present invention relates to a gastric electrical stimulation system, especially a micro-needle with ultrasonic capsule for a gastric electrical stimulation system.

### 2. Description of the Prior Art

Patients with diabetes mellitus (DM) often suffer from gastrointestinal disorders, if occur in the stomach, such disorders may include symptoms of “diabetic gastroparesis.” Now, the pathogenic mechanism of diabetic gastroparesis is still not very clear, but normally symptoms typically include easily satiety, abdominal bloating, nausea, vomiting, abdominal pain, and weight loss.

Current clinical speculation is that it is probably due to diabetic autonomic neuropathy (DAN) caused by hyperglycemia in diabetes, leading to abnormal gastrointestinal motility (enterogastric peristalsis dysmotility) in diabetic patients, this affects the enterogastric peristalsis, causing dysmotility, and can occur in any region of the gastrointestinal tract, potentially leading to severe seriously intestinal obstruction.

Clinically, prokinetic agents that promote gastrointestinal motility are often used to improve symptoms of gastroparesis. However, long-term use of these drugs can also lead to adverse drug reactions (ADR), significantly affecting the patient's life when symptoms of diabetic gastroparesis are seriously severed. Now, gastric electrical stimulation (GES) therapy has become a quite effective method for treating diabetic gastroparesis clinically, that is, the previous gastric electrical stimulation (GES) therapy involves the use of an endoscope or surgery to implant electrodes into the greater curvature of the stomach, with the battery of device placed subcutaneously, delivering electrical stimulation at 5 milliamps per minute and 14 hertz.

Current commercially available devices require invasive surgery for implantation, this form of neurostimulation treatment for gastroparesis can only be administered by physicians through long-term and repetitive surgical procedures, offering low-energy electrical stimulation through a single channel. Not only is there no access to real-time clinical live imaging of the stomach, but the treatment process can also easily cause physical discomfort for patients.

### SUMMARY OF THE INVENTION

In view of the above-mentioned description, the purpose of the present invention is to overcome the shortcoming of existing technique, proposes the present invention pertains to a gastric electrical stimulation system that utilizes implantable gastric stimulation technology integrated within an ultrasonic capsule. By employing electrodes and a pulse generator for electrical stimulation of the stomach tissues, the present invention effectively alleviates the symptoms of gastroparesis, thereby improving the quality of life for patients.

The gastric electrical stimulation system of the present invention includes a transmission device and a reception device.

In the gastric electrical stimulation system of the invention, the transmission device comprises the following components: a signal inputting component, a first processor, a display, and a first radio-frequency (RF) antenna. The signal inputting component is electrically connected to the first processor, which is in turn electrically connected to the signal display. The first processor is also electrically connected to the first RF antenna.

In this gastric electrical stimulation system of the invention, the reception device includes the following components: electrodes, a first voltage device, a second voltage device, a port, a second processor, and a second RF antenna. The electrodes are electrically connected to the first voltage device, which is connected to the second voltage device. The second voltage device is connected to the port, which is connected to the second processor, and the second processor is connected to the second RF antenna.

The gastric electrical stimulation system of this invention, which featuring a piezoelectric receiver, includes: an antenna, a transmitter, a button cell battery, a micro implantable field-programmable gate array (FPGA) controller, a specific application integrated circuit printed circuit board, a pad, a band-pass filter, an injection system, stimulation sensors, a printed circuit board, sensors, filters, and stimulating microneedles.

An advantage of this gastric electrical stimulation system of the invention is that having charging unit, which is capable of generating an adjustable power output between positive 20 decibels-milliwatts (dBm) and positive 30 dBm, the invention possesses sufficient capacity to maintain a constant rectified voltage for the capsule under conditions of body movement and gastric dynamics.

Another advantage of this gastric electrical stimulation system of the invention is that, since the capsule main body needs to be exposed to an electromagnetic wireless power transmission (WPT) environment, the invention must comply with restrictions on radio frequency (RF) energy absorption to meet health and safety standards in medical devices.

An advantage of this gastric electrical stimulation system of the invention, which is capable of integrating real-time bioelectrical activity signals and imaging for monitoring the gastric system, and offering a capability not currently available on the market, providing multi-channel electrical stimulation at both low and high energy simultaneously, in order to achieve the objective of freely regulating capsule activity.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a schematic diagram of the gastric electrical stimulation system of the present invention.

FIG. 2A is a schematic diagram of the piezoelectric receiver (ultrasonic capsule) of the present invention.

FIG. 2B is a schematic diagram of the piezoelectric receiver (ultrasonic capsule) of the present invention.

FIG. 2C is a circuit diagram of the piezoelectric receiver (ultrasonic capsule) of the present invention.

FIG. 3A illustrates a method for forming the capsule-type suction cup fish electrode shell of the present invention.

FIG. 3B is a schematic diagram of the capsule-type suction cup fish electrode shell of the present invention.

FIG. 3C is a schematic diagram of the capsule-type suction cup fish electrode shell of the present invention.

### DESCRIPTION OF THE PREFERRED EMBODIMENT

The attached figures should be used to describe the implement way of the present invention. In the figures, the same element symbol is used to represent the same element, in order to describe the element more clearly, its size or thickness might be scaled.

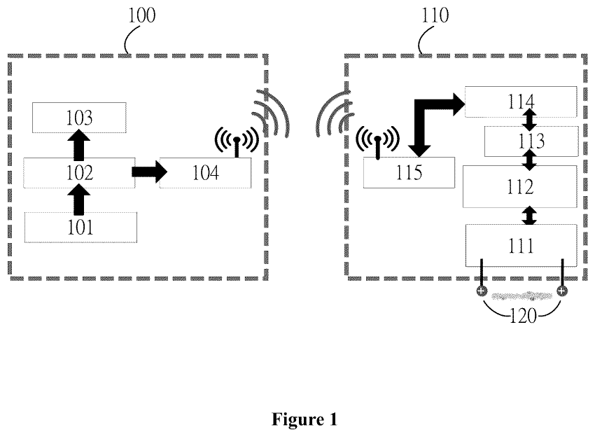

As shown in FIG. 1, the gastric electrical stimulation system of the present invention comprises a transmission device 100 and a reception device 110.

As shown in FIG. 1, the gastric electrical stimulation system of the present invention, wherein the transmission device 100 comprises the following components: a signal inputting component 101, a first processor 102, a display (LCD) 103, and a first radio frequency antenna 104, and, the signal inputting component 101 is electrically connected to the first processor 102, the first processor 102 is electrically connected to the display 103, and the first processor 102 is also electrically connected to the first radio frequency antenna 104.

Continuing with FIG. 1, in the gastric electrical stimulation system of the present invention, the first processor 102 is a micro-implantable field-programmable gate array (FPGA) master control chip.

Further, still as shown in FIG. 1, the present invention involves transmitting control commands or the relevant data from the transmission end of the first radio frequency antenna 104 to the reception end of the signal inputting component 101, at this time, transmitting control commands or the relevant data are devoted by the signal inputting component 101, and corresponding control operations are executed by the first processor 102. The relevant data comprises information on the internal structure of the digestive tract, i.e., including rebound strength, echo time, etc. Subsequently, the following ultrasonic capsule will transmit the relevant data externally.

As depicted in FIG. 1 for the gastric electrical stimulation system of the present invention, the reception device 110 includes the following components: an electrode 120, a first voltage regulator 111, a second voltage regulator 112, a port 113, a second processor 114, and a second radio frequency antenna 115, the electrode 120 is electrically connected to the first voltage regulator 111, the first voltage regulator 111 is electrically connected to the second voltage regulator 112, the second voltage regulator 112 is electrically connected to the port 113, the port 113 is electrically connected to the second processor 114, and the second processor 114 is electrically connected to the second radio frequency antenna 115.

At this moment, as still depicted in FIG. 1 of the gastric electrical stimulation system of the present invention, the second radio frequency antenna 115 can receive the drive signal transmitted by the transmission device 100, and then, the second radio frequency antenna 115 is also used to receive the rebounded ultrasonic signals. When the ultrasonic signal encounters tissues, organs, or abnormal areas within the digestive tract, part of the ultrasonic signal is absorbed, while the remainder is reflected back at the suitable time.

As continued in FIG. 1, in the gastric electrical stimulation system of the present invention, the second processor 114 is a micro-implantable field-programmable gate array (FPGA) master control chip.

Still shown in FIG. 1, the gastric electrical stimulation system of the present invention comprises the first processor 102 (a micro-implantable field-programmable gate array master control chip) in the transmission device 100, utilizing a transmission module capable of generating frequencies of 868 to 915 megahertz (MHz), through the first radio frequency antenna 104, transmitting to the reception device 110 in FIG. 1A, the reception device 110 is powered by a button battery 3.0 volts (V) and is easily transferable to the human intestinal tract.

Furthermore, the gastric electrical stimulation system of the present invention as shown in FIG. 1, the reception device 110 and the transmission device 100 are composed of the same modules, through an NPN-type transistor (with the specifications in this invention using an NPN-type transistor for switching clock rates), the operating frequencies of timer are 14 Hz, 28 Hz, and 55 Hz, with pulse widths from 1 microsecond to 999 microseconds, the corresponding clock-ratio ranges from 0.0014% to 5.208%, and the switching-time-cycle is between 0.1 seconds to 9.9 seconds.

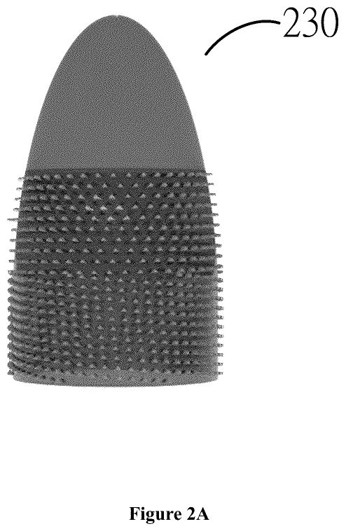

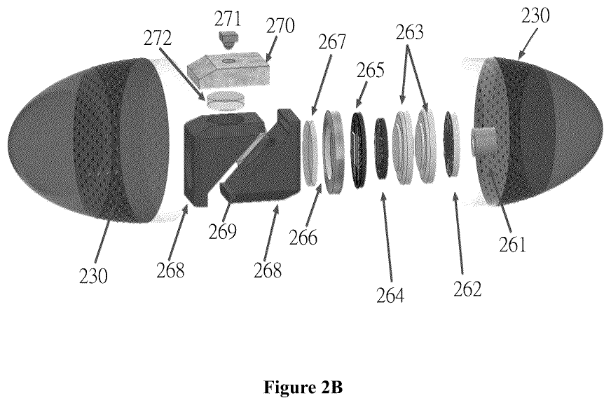

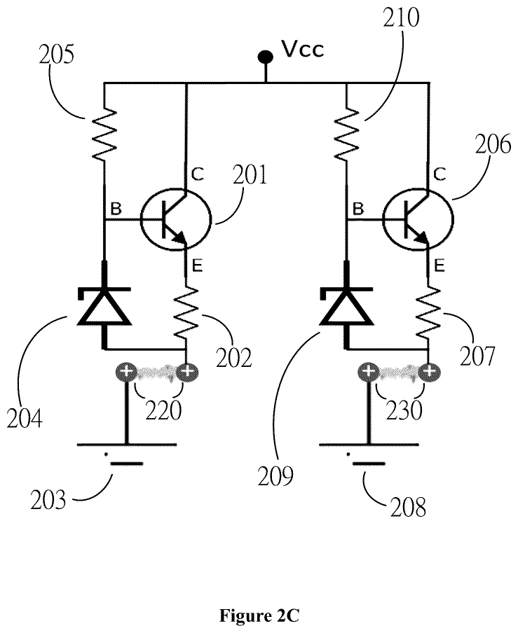

As illustrated in FIG. 2A, the piezoelectric receiver (ultrasonic capsule) of this invention is designed following the concept of Remora's suckerfish, incorporating a miniaturized needle-equipped ultrasonic capsule 230, is encapsulated within a silicone sleeve, offering waterproof protection. The invention adopts electrodes designed based on Remora's suckerfish, and utilizes Polydimethylsiloxane (PDMS), using microneedles (MN), employing a method of a gelatin microneedle drug patch, creating the brand new type of micro-needle-equipped ultrasonic capsule 230.

FIG. 2B shows a schematic of the piezoelectric receiver (ultrasonic capsule) of the invention, including: an antenna 261, transmitter 262, button battery 263, micro-implantable field-programmable gate array (FPGA) controller 264, application-specific integrated-circuit printed circuit board (ASIC PCB) 265, spacer 266, bandpass filter 267, injection system 268, stimulation sensor 269, printed circuit board (PCB) 270, transducer 271, filter 272, and the micro-needle-equipped ultrasonic capsule 230 with stimulation microneedles.

In the schematic of the piezoelectric receiver (ultrasonic capsule) shown in FIG. 2B, the antenna 261 receives the driving signal transmitted by the transmission device 100, and then the transmitter 262 sends the drive signal to the controller 264. The piezoelectric receiver (ultrasonic capsule) of the invention converts the received ultrasonic signals into electrical signals. These electrical signals contain information about the internal structures of the gastrointestinal tract, including data on the rebound strength, echo time, etc. The signals are then output through the capacitive voltage divider of the radio frequency power amplification circuit 265, wherein both the radio frequency power amplification circuit 265, and the capacitive voltage divider are located on the aforementioned application-specific integrated-circuit printed circuit board 265 on a multi-layered circuit board, thus marked on the same item. The data transmitted can be received by the transmission device 100, and the embedded envelope detector (envelope demodulation) 270 performs the demodulation, wherein the embedded envelope detector 270 is situated on the aforementioned printed circuit board 270 on a multi-layer circuit board, marked as the same item.

As depicted in FIG. 2B, the piezoelectric receiver (ultrasonic capsule) of the invention has a power consumption of 20 milliwatts (mW). When operated with a custom button battery 263, the custom button battery 26 can provide 3 volts (V) of electricity and work with 620 milliampere-hours (mAh), allowing continuous operation for over 2 hours.

Building on the aforementioned, FIG. 2B illustrates the piezoelectric receiver (ultrasonic capsule) of the invention, which utilizes the receiving capsule shell made of hard ceramic material, also, which can help provide higher collection power and collection efficiency, and wherein, the design of the circuit specifications is based on the use of a microprocessor, which has a narrow-band capacitor network to match the coil to 50 watts (W), and then, through a radio frequency power amplifier (radio frequency power amplifier), by using the 13.56 MHz Radio Frequency Identification (RFID) system, which uses self-manufactured microchip (Microprocessor) products for driving RF output.

In addition to the foregoing, FIG. 2B is a schematic diagram of a piezoelectric receiver (ultrasonic capsule), which has a synchronous buck-boost DC/DC converter with a secondary circuit, directly powering the radio frequency power amplifier, by increasing (or decreasing) the feedback loop on this synchronous buck-boost DC/DC converter, i.e., the model LTC3111 is for the synchronous buck-boost DC/DC converter of this invention, the programmable digital potentiometer chip of the present invention can be used to adjust the corresponding increased or decreased for the wireless power outputting to the capsule, in order to achieve the establishment of a constant wireless capsule-type power supply system. The present invention is a kind of collection-power of the load resistance and the working frequency function of the capsule piezoelectric receiver under the incident sound pressure of approximately 40 kPa, and then transmits corresponding control commands to the capsule processor controlled by itself or the circuit device equipment controlled by itself.

Continuing from the previous description, FIG. 2B is a schematic of the piezoelectric receiver (ultrasonic capsule). The operating frequencies of its timer are 14 hertz (Hz), 28 hertz (Hz), and 55 hertz (Hz), respectively, with pulse widths from 1 microsecond (us) to 999 microseconds (us). The corresponding switch timing is verified by a system (voltage/current) with cycles from 0.1 to 9.9. Finally, using wireless-type, and through the implantation of hard piezoelectric ceramics under the skin, the power management circuit is completed, achieving a charging current of 3 milliamperes (mA). With 3 milliampere-hours (mAh), the charging battery can wirelessly charge the ultrasonic capsule within approximately 1 hour, transmitting the corresponding control commands to the capsule processor controlled by itself and circuit device equipment controlled by itself.

As shown for the circuit diagram of the piezoelectric receiver of the present invention in FIG. 2C, the piezoelectric receiver is installed inside the ultrasonic capsule, which has functions to receive electrical stimulation messages, including the first NPN transistor 201 is electrically connected to the first resistor 202, the first resistor 202 is electrically connected to the first battery 203, the first battery 203 is electrically connected to the first diode 204, the first diode 204 is electrically connected to the second resistor 205. Additionally, the second NPN transistor 206 is electrically connected to the third resistor 207, the third resistor 207 is electrically connected to the second battery 208, the second battery 208 is electrically connected to the second diode 209, the second diode 209 is electrically connected to the fourth resistor 210, wherein the second resistor 205 and the fourth resistor 210 are connected in series.

As depicted in the circuit diagram of the piezoelectric receiver of the present invention in FIG. 2C, at this point, the piezoelectric receiver of the invention converts the received ultrasonic signals into the electrical signals, these electrical signals contain the relevant data about the internal structure of the digestive tract, including the information like the intensity of the rebound and the echo timing.

FIGS. 2A and 2B are schematic diagrams of the piezoelectric receiver (ultrasonic capsule). In fact, the present invention requires an efficient and adaptable micro ultrasonic capsule that can operate freely in the harsh environment of the stomach and intestines. Therefore, the invention adopts a design method similar to Remora suckerfish. Although voltage control and current control stimulation pulses can be used to modulate the electrophysiological activities of the gastrointestinal tract, uniform voltage distribution may not be possible on the stimulation electrodes in the stomach due to heterogeneous biological impedance in the gastrointestinal tract and the impedance at the electrode-tissue interface that changes over time. It is important to note that in current-controlled stimulation, providing a constant charge for each pulse can minimize problems in future usage to the greatest extent.

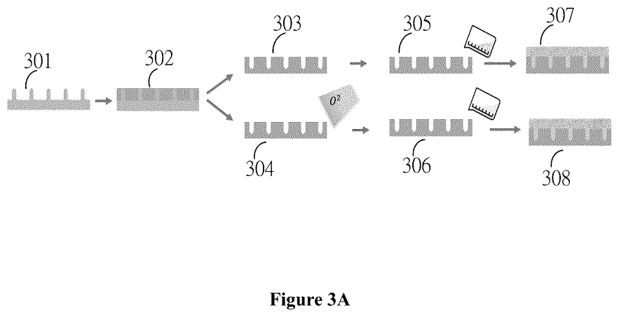

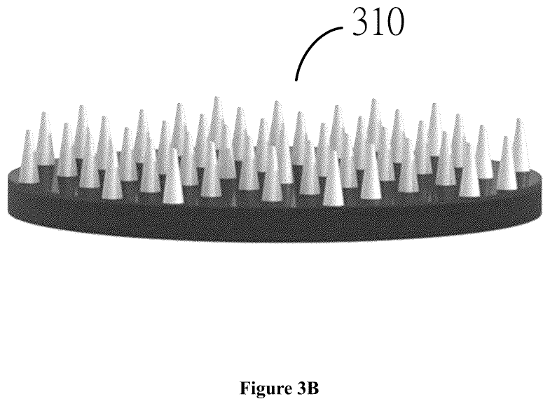

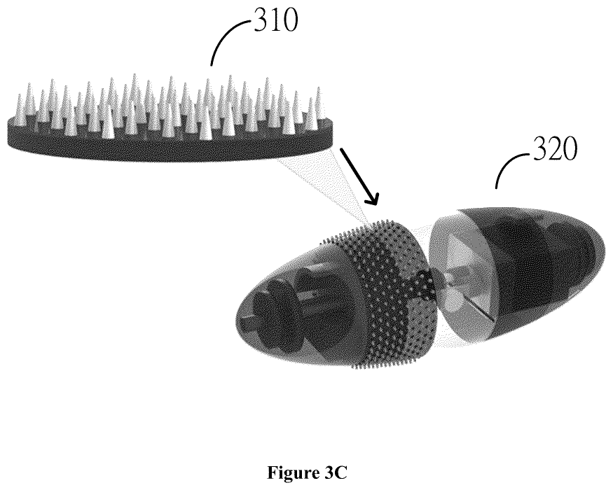

As shown in FIG. 3A, the method for forming the shell of the capsule-type bionic electrode of the present invention, the present invention basically involves using the mixture of powdered polydimethylsiloxane (PDMS) gel, Rhodamine 6G, Lissamine Green B dye (LGB), and magnetic particles. This mixture is first applied to form conical foot structures (serving as the capsule-type suckerfish electrodes) and then, during the solidification process, an external magnetic field is applied. After the structure is peeled off from the base substrate, Remora suckerfish's electrode, approximately 17 millimeters (mm) long, 7 millimeters (mm) wide, and 150 micrometers (μm) thick, is obtained.

As shown in FIG. 3A, the method for forming the capsule-type suckerfish electrode, the first step 301 involves using a microneedle patch as a template, that is, using the conventional microneedles as a mold.

As shown in FIG. 3A, the method for forming the capsule-type suckerfish electrode, the next step 302 is to pour dimethylsiloxane solution over the aforementioned microneedle patch template, and then heat-cure the polydimethylsiloxane (PDMS) mold.

As shown in FIG. 3A, the method for forming the capsule-type suckerfish electrode, the subsequent step 303 is to invert the aforementioned PDMS mold so that the microneedles are pointing downwards.

As shown in FIG. 3A, the method for forming capsule-type Remora suckerfish's electrode, then in step 305, involves pouring a 10% gelatin solution (gelatin sheet) containing medication into the pure dimethylsiloxane (PDMS) mold, the gelatin solution is dried at room temperature for about 2 hours.

As shown in FIG. 3A, the method for forming a capsule-type suckerfish electrode, next in step 307, results in the formation of a first combination part and a second combination part composed of dimethylsiloxane and gelatin microneedle patches. Since the invention uses biocompatible silicone, which is highly compatible with biological bodies, less likely to cause allergies or rejection reactions, as the suitable material for the combination of dimethylsiloxane and gelatin microneedle patches.

As shown in FIG. 3A, the method for forming a capsule-type suckerfish electrode initially involves step 301, using microneedle patches as templates, that is, using conventional microneedles as molds.

As shown in FIG. 3A, the method for forming a capsule-type suckerfish electrode, then in step 302, involves pouring dimethylsiloxane solution onto the aforementioned microneedle patch template, followed by heating to solidify the dimethylsiloxane (PDMS) mold.

As shown in FIG. 3A, the method for forming a capsule-type suckerfish electrode, then in step 304, involves inverting the aforementioned dimethylsiloxane mold so that the microneedles are directed downward, and performing O₂ plasma treatment to enhance the curing effect on the dimethylsiloxane mold, thereby increasing the adhesive force between the microneedle patch and the dimethylsiloxane layer.

As shown in FIG. 3A, the method for forming a capsule-type suckerfish electrode, then in step 306, involves pouring a 10% gelatin solution (gelatin) loaded with drugs into the pure dimethylsiloxane mold. The gelatin solution is dried at room temperature for about 2 hours.

As shown in FIG. 3A, the method for forming a capsule-type suckerfish electrode, next at step 308, involves pouring the gelatin solution into the mold and drying the gelatin solution at room temperature, repeating three times, to form a third combination component consisting of O₂ plasma-treated dimethylsiloxane and gelatin microneedle patches. Here, the invention uses biocompatible silicone and cross-linking agents to form a three-dimensional network structure. This is primarily because cross-linking agents can establish chemical bonds between siloxane monomers, making the silicone more robust, durable, and possessing better physical properties.

FIG. 3B illustrates the outer shells 310 and 320 of the capsule-type suckerfish electrode, formed by the aforementioned steps in FIG. 3B. Notably, Rhodamine 6G and Lissamine Green B dye (LGB), each at a concentration of 0.5 mg/ml, which can be added to the gelatin solution as model drugs. Lissamine Green B dye (LGB) is a dye that can be mixed into the gelatin solution used for visualizing the delivery of microneedle patches, serving to identify the mode of drug delivery and the pathways to the pathological area through the microneedle patches.

As shown in FIG. 3C, the outer shells 310 and 320 of the capsule-type suckerfish electrode can ultimately be observed through a Scanning Electron Microscope (specifically, the SU8230 model produced by HITACHI in Japan) to examine the morphology of the multi-layer suckerfish electrode attached to the outer surface area of the ultrasound capsule. The main body of the ultrasound capsule and the nozzle mold are made by using polylactic acid (PLA) and a 3D printer, and by utilizing an Electromagnetic Actuation (EMA) system composed of six coils. This system generates a uniform gradient magnetic field based on the current passing through the coils, confirming that the capsule-type suckerfish electrode has been successfully attached to the outer wall of the microneedle ultrasound capsule.

This invention relates to a gastric electrical stimulation system that integrates implantable gastric electrical stimulation capsule technology with ultrasound capsules. By utilizing electrodes and a pulse generator for electrical stimulation of the stomach tissue, the present invention can effectively alleviate symptoms of gastric atony, thereby improving the quality life of patient. One advantage of this gastric electrical stimulation system includes a charging unit capable of generating an adjustable power between positive 20 dBm and positive 30 dBm, enough sufficient capacity to operate with body movement and gastric motility, providing a constant rectified voltage for the capsule. However, since the capsule's main body of this invention needs to be exposed to a Wireless Power Transfer (WPT) environment, comply with the restrictions on Radio Frequency (RF) energy absorption to meet health standards in medical equipment. Furthermore, this invention pertains to an operational type of gastric electrical stimulation system, capable of integrating real-time bioelectrical activity signals and images of the gastric system. The invention can achieve currently available on the market for a multi-channel system providing both low-energy and high-energy electrical stimulation simultaneously, allowing for the free adjustment of capsule activity.

The above description is only the preferred embodiment of this invention and is not intended to limit the patent application scope of this invention. Any other equivalent changes or modifications, which are made without departing from the spirit of this invention, should be included within the scope of the following patent application.

It is understood that various other modifications will be apparent to and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

What is claimed is:

1. A gastric electrical stimulation system, comprising:
a transmission device; and
a reception device.

2. The gastric electrical stimulation system according to claim 1, wherein said transmission device comprises:
a signal inputting component;
a first processor;
a display; and
a first RF antenna, wherein said signal inputting component being electrically connected to said first processor, said first processor being electrically connected to said display, and said first processor is electrically connected to said first RF antenna.

3. The gastric electrical stimulation system according to claim 1, wherein said first processor comprises a miniature implantable field-programmable gate array (FPGA) master chip.

4. The gastric electrical stimulation system as claimed in claim 1, wherein said reception device comprises:
electrodes;
a first voltage regulator;
a second voltage regulator;
a port;
a second processor; and
a second RF antenna, wherein said electrodes being electrically connected to said first voltage regulator, said first voltage regulator being electrically connected to said second voltage regulator, said second voltage regulator being electrically connected to said port, said port being electrically connected to said second processor, and said second processor is electrically connected to said second RF antenna.

5. A piezoelectric receiver, comprising:
an antenna;
a transmitter;
a button cell battery;
a miniature implantable field-programmable gate array (FPGA) controller;
an application-specific integrated circuit (ASIC) printed circuit board;
a spacer;
a band-pass filter;
an injection system;
a stimulation sensor;
a printed circuit board;
a sensor;
a filter; and
a stimulating microneedle.
